(19) United States
(12) Patent Application Publication
(10) Pub. No.: US 2016/0129274 A1
(43) Pub. Date: May 12, 2016

(54) WEARABLE ENERGY DELIVERY SYSTEM

(71) Applicant: Sam Boong PARK, Saratoga, CA (US)

(72) Inventor: Sam Boong PARK, Saratoga, CA (US)

(21) Appl. No.: 14/537,354

(22) Filed: Nov. 10, 2014

(51) Int. Cl. A61N 2/00; A61N 2/02; A61N 1/04; A61F 7/02; A61N 1/36

(52) U.S. Cl. CPC A61N 2/002; A61F 7/02; A61N 1/36014; A61N 1/0484; A61N 2/02; A61F 2007/0233

(57) ABSTRACT

A wearable energy delivery system can be integrated with a wearable item to provide therapy to the user, including pain therapy, healing therapy, and muscle stimulation. Integration of the energy delivery system with apparel provides convenience to the user and allows hands free use of the system, which can be used during exercise or normal wear.

### BACKGROUND OF THE INVENTION

The present invention relates generally to energy delivery systems. More particularly, the invention relates to a wearable energy delivery system that operates while the user is engaged in physical activity.

Musculoskeletal pain is very common. In some cases, such pain is chronic and typically treated with drugs, such as ibuprofen or acetaminophen. Such drugs can be effective in managing mild to moderate pain, but chronic use of these drugs is associated with undesirable gastrointestinal and renal side effects as well as liver toxicity.

Delivery of energy (e.g., heat, magnetic, electromagnetic, infrared, ultrasonic, vibration, radio frequency, etc.) to problem areas of the body has been used to treat pain, and also to provide wound healing therapy. Electrostimulation has also been used for centuries for pain control. It is known that a static magnet has little effect on pain management and wound healing. However, pulsed electromagnetic energy can create an electrical potential within cells. Low-frequency pulsed electromagnetic fields (PEMF), at about 1000 Hz or less, have been used as therapy for chronic pain as well as wound and bone healing. PEMF is within the developing field of biomagnetics, which also includes transcutaneous electrical nerve stimulation (TENS). TENS is the use of an electric current to provide nerve stimulation for therapeutic purposes, and has been used to treat pain. Massage has also been used to treat pain, reduce anxiety and depression, and temporarily reduce blood pressure.

Although drug therapy and PEMF therapy can be effective in managing pain, there are continuing efforts to develop new devices that are not only effective for pain relief but also are provide convenience to the user for managing pain as well as firming muscles and enabling the user to be enhance athletic performance.

### SUMMARY OF THE INVENTION

In accordance with an embodiment, a wearable pulsed electromagnetic energy delivery device is provided. The wearable energy delivery device includes an article of clothing, an energy delivery head and a power supply unit. The energy delivery head is configured to generate a pulsed electromagnetic field (PEMF), and the energy delivery head is integrated with the article of clothing. The power supply unit is arranged to be carried by the article of clothing. At least one wire couples the power supply unit to the energy delivery head, and the wire is integrated with the article of clothing. The wearable pulsed electromagnetic energy delivery device is arranged to deliver therapeutic pulsed electromagnetic energy to a wearer of the article of clothing without requiring power from a device not carried by the article of clothing.

In accordance with another embodiment, a system is provided. The system includes a wearable pulsed electromagnetic energy delivery device and a mobile user interface. The wearable pulsed electromagnetic energy delivery device includes an energy delivery head configured to generate a pulsed electromagnetic field (PEMF) and a power control unit arranged to generate an electrical waveform used by the energy delivery head to generate the pulsed electromagnetic field. The wearable pulsed electromagnetic energy delivery device is arranged to deliver therapeutic pulsed electromagnetic energy to a wearer of the pulsed electromagnetic energy delivery device without requiring power from an external device not carried by the pulsed electromagnetic energy delivery device. The mobile user interface is executable on a computing device separate from the wearable device, and the mobile user interface is arranged to utilize resources of the computing device to communicate wirelessly with the power control unit and is configured to permit a user to direct operation of the wearable pulsed electromagnetic energy delivery device via the computing device.

In accordance with yet another embodiment, a wearable energy delivery device is provided. The wearable energy delivery device includes an energy delivery head and a power control unit. The energy delivery head is configured to emit at least two different types of energy selected from the group consisting of pulsed electromagnetic field (PEMF) energy, pulsed electrical energy, and heat energy. The power control unit is operably connected to the energy delivery head by at least one wire. The energy delivery head and the wire are attached to an article of clothing.

### BRIEF DESCRIPTION OF THE DRAWINGS

The invention, together with further objects and advantages thereof, may best be understood by reference to the following description taken in conjunction with the accompanying drawings in which:

FIG. 1 is a schematic diagram of a wearable energy delivery system in accordance with an embodiment.

FIG. 2A is a top view of an embodiment of an energy delivery head.

FIGS. 2B and 2C are side cross-sectional view embodiments of an energy delivery head.

FIG. 2D is a top view of another embodiment of an energy delivery head.

FIG. 3A is a plan view of the working surface of an energy delivery head of a wearable energy delivery system in accordance with an embodiment.

FIG. 3B is a side view of the energy delivery head of shown in FIG. 3A

FIGS. 4A-4D show different embodiments of the energy delivery head showing the working surface.

FIGS. 5A-5D show different configurations of an energy delivery head having a PEMF coil and another energy source nested within one another.

FIGS. 6A-6C are perspective views of different embodiments of the energy delivery head showing flex points or strips.

FIG. 6D is a side view of the embodiment shown in FIG. 6C.

FIG. 7A is a top view of an energy delivery head having a PEMF coil stacked over electrodes in accordance with an embodiment.

FIG. 7B is a side view of the energy delivery head shown in FIG. 7A.

FIG. 7C is a bottom view of the energy delivery head shown in FIGS. 7A and 7B.

FIG. 8 shows different embodiments of power control units and energy delivery heads on a variety of wearable items.

FIG. 9 shows some exemplary locations for energy delivery heads on a variety of wearable items.

FIG. 10 shows an example of decorative wiring in clothing.

FIGS. 11A-11E show different embodiments of exemplary energy delivery patterns.

It is to be understood that, in the drawings, like reference numerals designate like structural elements. Also, it is understood that the depictions in the figures are diagrammatic and not to scale.

### DETAILED DESCRIPTION OF THE PREFERRED EMBODIMENTS

The present invention relates generally to making devices for personal well being, specifically the use of an energy delivery system for pain management therapy, firming and toning of skin and muscles, and enhanced athletic performance, and apparatuses therefor.

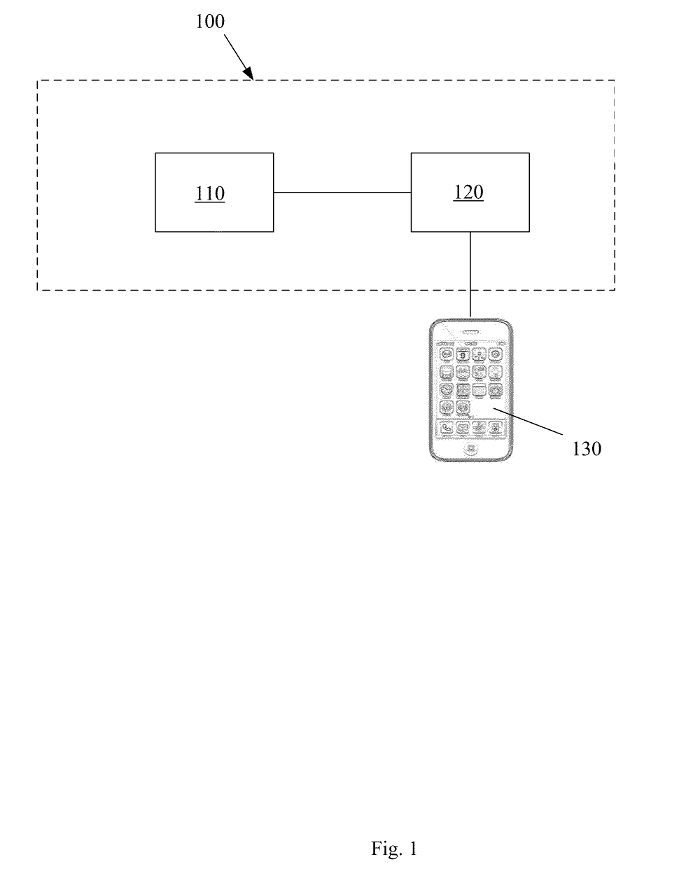

Referring initially to FIG. 1, a wearable energy delivery system 100 in accordance with one embodiment of the invention will be described. In the illustrated embodiment, a wearable energy delivery system 100 includes an energy delivery head 110 and a power control unit 120. A user interface 130, which can be part of the wearable energy delivery system 100 or on a separate device can be used to control the power control unit 120, which controls the energy delivery head 110. In FIG. 1, the user interface 130 is shown as being on a smartphone. However, it will be understood that the user interface 130 can be on any type of device, such as a computer, tablet, a user interface unit dedicated to the system 100, or even on the power control unit 120 or the energy delivery head 110.

The energy delivery head 110, which is integrated with a wearable item (e.g., an article of clothing, such as a shirt, pants, shorts, bra, hat, shoe, wristband, watch, socks, etc.), delivers energy, such as PEMF, energy sensory pulse or electrical sensory pulse (ESP), TENS, electrical muscle stimulation (EMS), neuromuscular electrical stimulation (NMES), static magnetic, ultrasonic, percussion, radio frequency (RF), vibration, and heat (near and far infrared) energy (or any combination thereof) to problem or target areas of the user's body. The energy delivery head 110 is powered by the power control unit 120 and controlled using the user interface 130. The energy delivery head 110 can be provided in any location in the wearable item such that it delivers energy located to the corresponding part of the body that requires pain relief, cellular repair, energy balancing, better blood circulation, etc. For example, the energy delivery head 110 can be integrated with a wearable item, such as a shoe or sock to provide therapy to feet. Alternatively, the energy delivery head 110 can be integrated in running tights or shorts to provide muscle stimulation or pain relief to legs during exercise. As noted above, the energy delivery head 110 can also be integrated with other wearable items, such as wrist bands, shirts, bras, etc.

In the embodiments described herein, the wearable energy delivery system 100 is capable of delivering one or more types of energy (alone or in combinations) to provide healing and/or pain therapy to affected areas of the body. A combination of different energies can provide short-term pain relief, long-term pain relief, surface healing, shallow depth healing, and/or deep healing depending on the energy combinations and configuration of the wearable energy delivery system 100. Each type of energy can be chosen for its unique healing property and different types of energies can be combined to provide multiple modes of healing and/or pain management with one or more energy therapies. The wearable energy delivery system 100 can be used for many different applications, including firming and tightening of skin and muscles, especially in the gluteal, abdominal, and pectoral muscles, pain relief, massage, muscle soreness therapy, wound therapy, healing therapy, and energizing muscles.

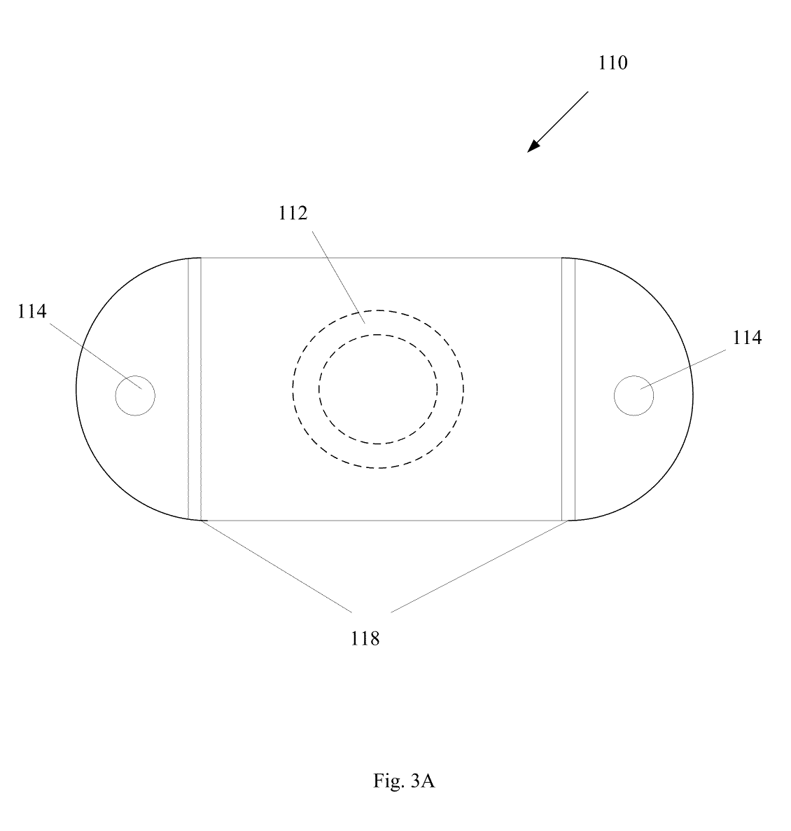

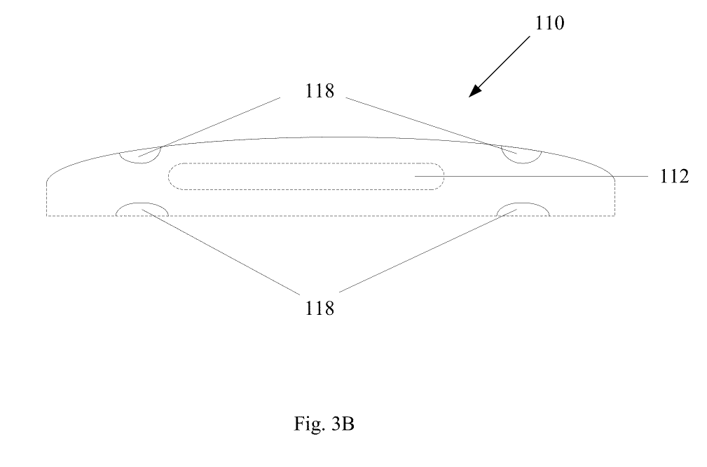

According to an embodiment, the energy delivery head 110 is capable of delivering at least two different types of energy simultaneously to target areas of the user's body. For example, in the embodiment illustrated in FIGS. 3A and 3B, the energy delivery head 110 can simultaneously deliver PEMF as well as another type of energy, such as, for example, TENS. In some embodiments, the user can use the user interface 130 to choose whether the energy delivery head 110 delivers one type of energy or more than one type of energy (and different combinations thereof). In the illustrated embodiment, PEMF can provide healing energy and better blood circulation to muscle tissue for long term benefits, whereas TENS therapy can provide quick pain relief by overwhelming nerve signals and blocking pain.

As noted above, PEMF therapy can provide healing energy to the tissues of the affected area. The United States Food and Drug Administration (FDA) has approved the use of PEMF for bone healing, trans-cranial magnetic therapy, and pain therapy. PEMF is provided without any sensation to the patient and PEMF can provide deep tissue relief when sufficient intensity of PEMF is delivered. TENS can be used to block the pain, but can also be used to heal the tissue if a sufficient intensity of TENS energy is delivered to the affected area.

Combining PEMF and TENS allows for delivery of both quick and long lasting therapy to the affected area. Energy delivery can be staggered between the energy sources or they can be delivered simultaneously (or a combination thereof) depending on the desired therapy needs. The quick action pain relief provided by TENS along with the longer-term cellular repair from PEMF can provide very effective treatment. Depending on the energy density of each of the energy sources, different depths of treatment is also possible. The combination of TENS and PEMF can provide therapy to deeper tissue without the excessive energy delivery required by the use of TENS alone (to provide deeper tissue relief), which could cause excessive muscle or nerve twitching.

Different energy sources provide therapy to different depths of the body tissue. For example, TENS and heat energy can provide therapy to more shallow areas, whereas PEMF can be used to penetrate to deeper tissue. Thus, a combination of PEMF with TENS or heat energy, for example, can provide both shallow and deep tissue therapy to the affected area during the same treatment session.

The time effect of the pain relief is also different for different energy sources. For example, TENS and heat energy typically provide immediate and short-term pain relief, whereas PEMF typically provides healing therapy to the cells that are causing the pain for actual repair of the cells. A combination of PEMF with TENS or heat energy, for example, can provide both short-term and long-term pain relief. Typically, TENS provides shallow therapeutic effects and PEMF can provide deeper therapeutic effects depending on the energy settings of each energy mode. In order for TENS alone to provide deep tissue therapy, the intensity of the energy (voltage and current) must be increased, which typically results in excess twitching of the muscles and nerves. PEMF can provide deep tissue therapy without adverse muscle and nerve effects.

If PEMF is combined with heat energy (near or far infrared), the heating effect will provide a soothing effect to skin and short-term relief, and PEMF will provide long-term healing therapeutic effects to the affected area. Typically, heat energy provides shallow therapeutic effects and, as noted above, PEMF can provide deeper therapeutic effects depending on the energy settings of each energy mode. Similar types of short and long-term benefits can be achieved by combining other types of energy, such as ultrasonic, percussion, radio-frequency, etc.

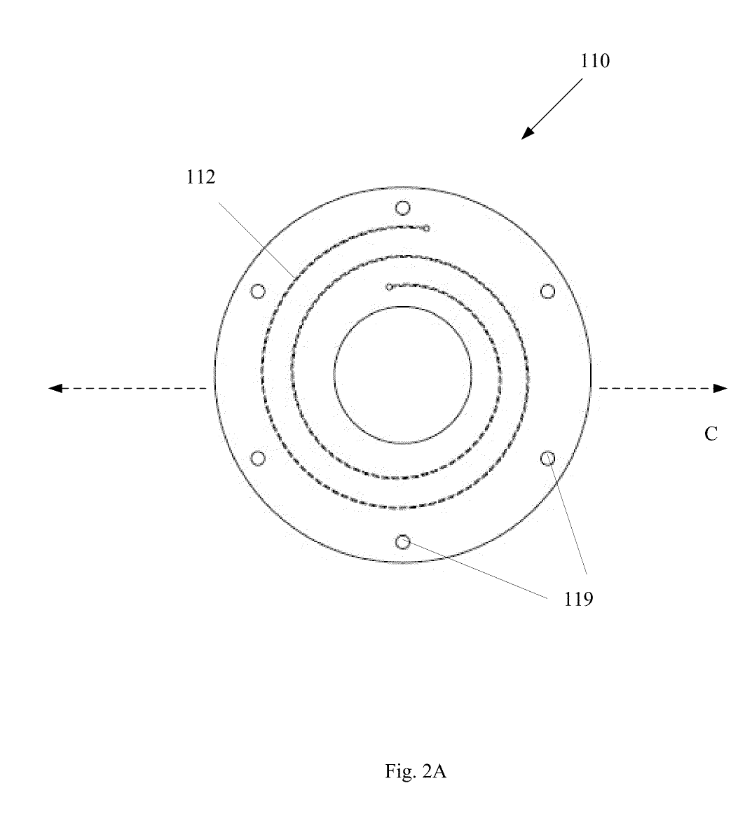

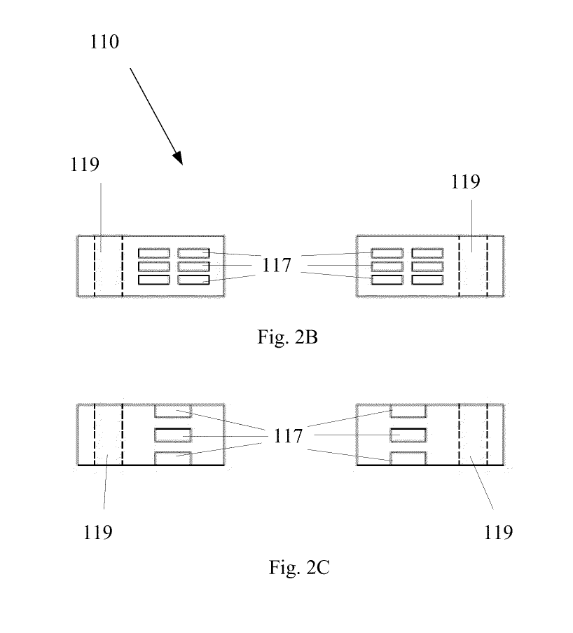

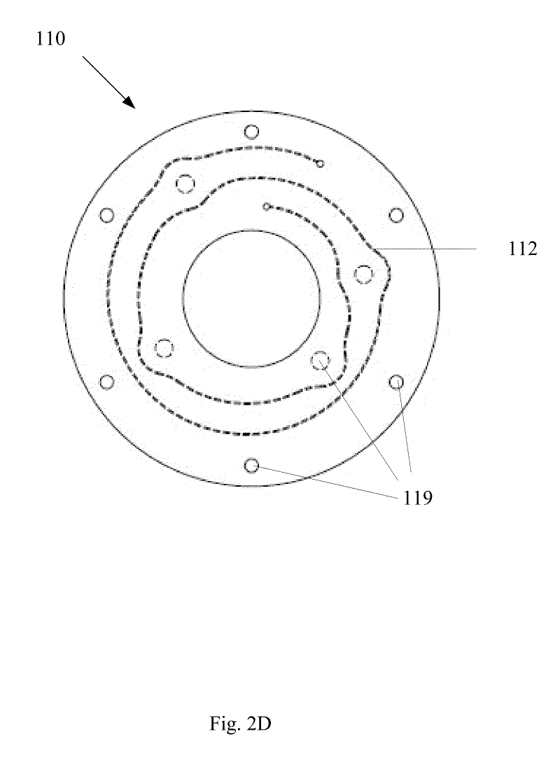

FIGS. 2A-2D show embodiments of an energy delivery head 110 formed from a flexible material, such as a flex circuit, flexible PC board, flexible wires, etc. An electromagnetic coil 112 can be embedded within the flexible material or can be on the surface of the energy delivery head 110. The energy delivery head 110 can be any size and formed of any number of layers of flexible material. Each flexible material layer can include an electromagnetic coil 112 that has one layer of trace or coil wire 117. Alternatively, each flexible material layer can have an electromagnetic coil 112 that has multiple layers of trace or coil wire 117, as shown in FIGS. 2B and 2C. Multiple flexible material layers can be bonded or laminated together to create a more powerful magnetic field. The advantage of using multiple flexible material layers is that the multiple thin layers are more flexible than a single thick flexible material layer having multiple layers of traces 117. Multiple flexible material layers can be bonded together to make one thick piece or multiple flexible material layers can be loosely fastened together for more flexibility of the energy delivery head 110. The traces or coil wire 117 can be embedded in the flexible material, as shown in FIG. 2B, or can be embedded in as well as on the surface of the flexible material, as shown in FIG. 2C.

As shown in FIGS. 2A-2D, multiple openings 119 can be provided as attachment points for the energy delivery head 110 to facilitate attachment of the energy delivery head 110 to the wearable item. For example, the openings 119 can be used to sew or otherwise attach the energy delivery head 110 to the wearable item. The openings 119 can be positioned anywhere on the energy delivery head 110. As shown in FIG. 2D, the openings 119 can even be positioned in a decorative pattern. The electromagnetic coil 112 can also be positioned to form a decorative pattern, as shown in FIG. 2D. The energy delivery head 110 can be attached to the wearable item in different ways. For example, the energy delivery head 110 can be sewn, laminated, snapped, or woven into the wearable item.

FIG. 3A is a plan view of the working surface of the energy delivery head 110 in accordance with an embodiment, and FIG. 3B is a side view of the energy delivery head 110 shown in FIG. 3A. As shown in FIG. 3A, the working surface, which contacts the skin (or clothing of the user), has an electromagnetic coil 112 and two other energy sources 114, such as electrodes. The electromagnetic coil 112 can be embedded within the energy delivery head 110. The user interface 130, which is operably connected to the energy delivery head 110, is used to activate the electromagnetic coil 112 to deliver the pulsed electromagnetic energy to the user. As discussed above, PEMF has been used for pain therapy as well as healing therapy.

However, PEMF cannot be sensed by the user so the user may not be aware that the PEMF is being delivered or the user may not believe that the PEMF is effective since it is not sensed by the user. Thus, a device that delivers only a type of energy, such as PEMF, that cannot be sensed by the user may be less effective because the user cannot feel the PEMF being applied, and therefore the user may not believe that the PEMF is working.

Thus, in the embodiments shown in FIGS. 3-5, the energy delivery head 110 can also deliver at least two different types of energy. In some embodiments, at least one of the energies is capable of being sensed by the user. For example, in addition to delivering an energy that cannot be sensed (e.g., PEMF), the energy delivery head 110 can emit an energy that can be sensed so that the user realizes the wearable energy delivery system 100 is working. Realizing that the system is working can provide the added benefit of a placebo effect, as described in more detail below.

For example, in the illustrated embodiment, the energy delivery head 110 can also deliver via electrodes 114 an energy that can be sensed, such as ESP or TENS, in addition to the PEMF from the electromagnetic coil 112. ESP, which can deliver electrical pulses at relatively low frequency, can provide a tingling sensation so that the user can feel the wearable energy delivery system 100 working, which can provide the benefit of a placebo effect. According to an embodiment the energy delivery head 110 delivers ESP at a frequency in a range of about 0.1-100 Hz. In this embodiment, the frequency is more preferably about 0.1-17 Hz, and even more preferably about 0.1-5 Hz.

Similarly, as TENS is electro-stimulation of the nerves, the user feels the delivery of TENS as opposed to PEMS, which does not provide any sensation to the user. In addition to the therapeutic benefits provided by TENS, there may also be the additional benefit of a placebo effect because the user can feel the TENS as opposed to not feeling the PEMF. According to an embodiment, the pulsed electrical energy (i.e., ESP or TENS) is pulsed between the pulses of the PEMF energy.

As noted above, an energy that can be sensed, such as ESP or TENS, can provide the benefit of a placebo effect. As ESP and TENS are felt by the user, the perception that the ESP or TENS is working may help a user achieve a sense of well-being, which may bring about a positive physical response in addition to the psychological response. Thus, the combined energy delivery can have not only a cumulative therapeutic effect from two (or more) different types of energies, but also a placebo effect if at least one of the energies is sensed by the user. Furthermore, both ESP and TENS require skin contact as opposed to PEMF, which does not require skin contact, so the mere contact of the energy delivery head 110 to the skin may provide a placebo effect.

In some embodiments, the wearable energy delivery system 100 provides indication that the wearable energy delivery system 100 is operating if the wearable energy delivery system 100 is delivering only PEMF or PEMF in combination with another energy that cannot be sensed so that the user knows that the wearable energy delivery system 100 is working (i.e., emitting energy). For example, the energy delivery head 110 can emit a light, whether blinking or not, or a sound to indicate that the wearable energy delivery system 100 is working. According to another embodiment, a display on the wearable energy delivery system 100 can provide an indication, such as a blinking heart or energy levels, that the device is working. Alternatively, a similar display on a separate device, such as a computer, smartphone, smart watch, tablet, etc., can provide such an indication. The user interface 130 for controlling the wearable energy delivery system 100 can be separate from the energy delivery head 110 and/or power control unit 120. In some embodiments, the user interface 130 is actually on another device, such as a computer, smartphone, smart watch, tablet, etc.

Regardless of the location of the user interface 130, it can be used for controlling the energy emitted from the energy delivery head 110. The user interface 130 can include a display, knobs, levers and/or buttons and the like to control the energy delivery head 110. In some embodiments, the display can be a touchscreen display and separate knobs, levers, and buttons may not be necessary. The user interface 130 can be used to, for example, turn on the wearable energy delivery system 100, select the energy or energies emitted from the energy delivery head 110, control the intensity of the energy or energies, and control the duration of the energy emission from the energy delivery head 110.

According to an embodiment, the system 100 has a “sleep” mode in which only an energy that cannot be sensed (e.g., PEMF) is delivered. A user can select sleep mode if he or she desires continued therapy while sleeping. It will be understood that, in sleep mode, it is desirable to deliver an energy that cannot be sensed so that the user can sleep without being disturbed by a sensation, such as a tingling sensation which can be provided by TENS. Thus, the energy delivery head 100 can be integrated with sleep apparel (i.e., pajamas).

In other embodiments, the energy delivery head 110 can deliver other combinations of different types of energies instead of or in addition to PEMF and TENS. Other types of energy that can be delivered by the energy delivery head 110 include: magnetic energy from a permanent magnet, heat energies, near and far infrared, percussion, ultrasonic, and vibration. Magnetic energy cannot be sensed by the user as opposed to percussion, ultrasonic, vibration, and heat energies.

The energy delivery head 110 can be configured in different ways to deliver energies in different ways. As shown in the illustrated embodiment of FIG. 3, for an energy delivery head 110 for emitting both PEMF and TENS, the electromagnetic coil 112 is positioned in the center of the working surface of the energy delivery head 110 and the electrodes 114 are positioned on both sides of the electromagnetic coil 112. The electromagnetic coil 112 delivers PEMF while the electrodes 114 deliver TENS to the user.

The configuration shown in FIG. 3 provides short and long term benefits, as described above. A wearable energy delivery system 100 having an energy delivery head 110 with the configuration shown in FIG. 3 can be placed around the affected area to provide combined therapy. TENS from the electrodes 114 can provide short-term relief from pain in the affected area in a larger scale and shallow tissue level due to current flow between the two electrodes 114. PEMF from the electromagnetic coil 112 located between the TENS electrodes 114 provides more concentrated and deeper therapy to the affected area that is simultaneously receiving pain relief from TENS.

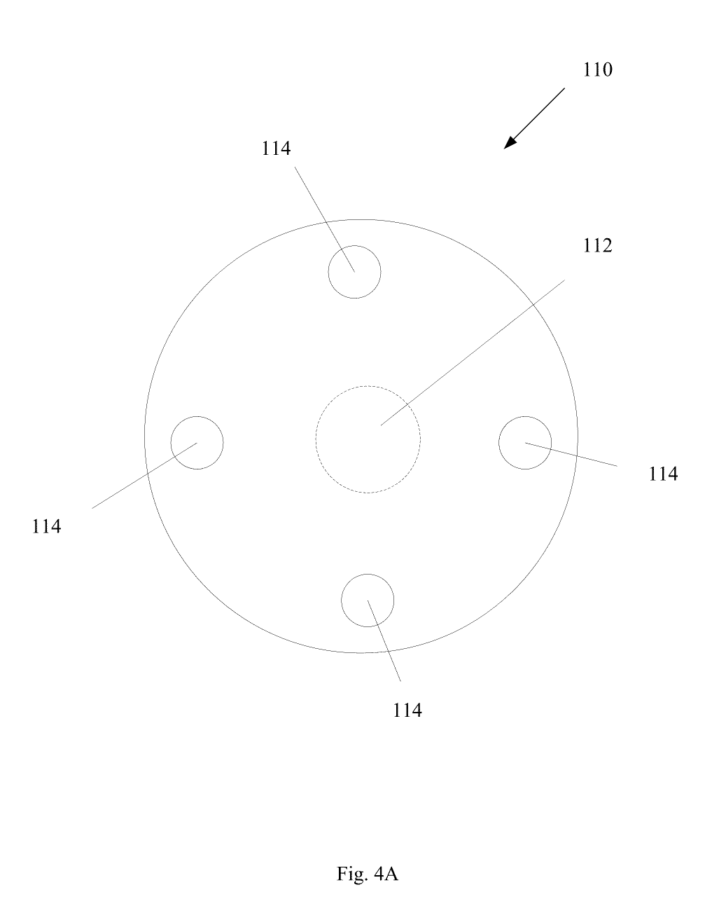

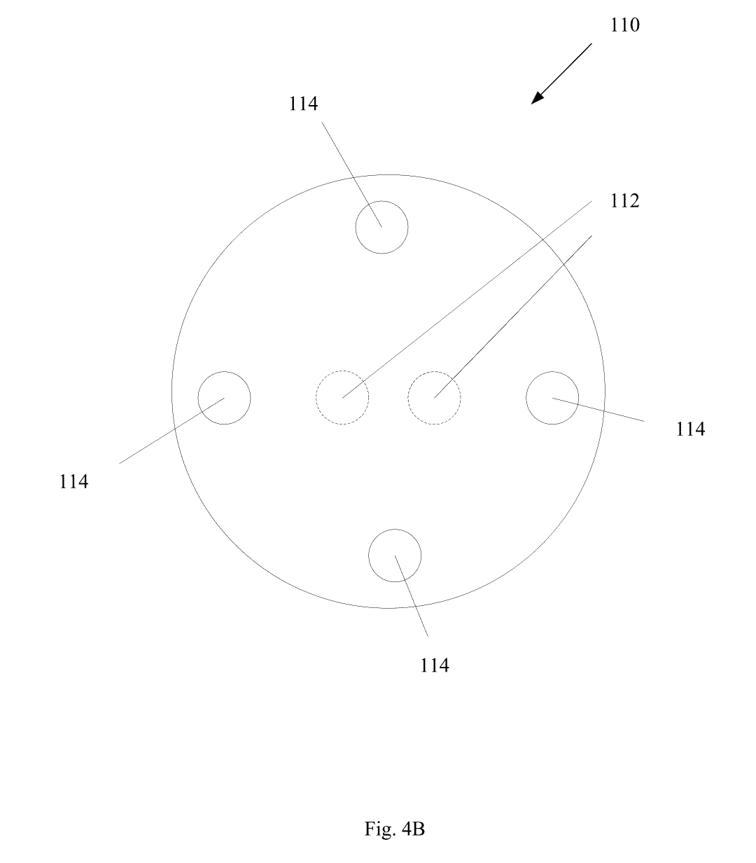

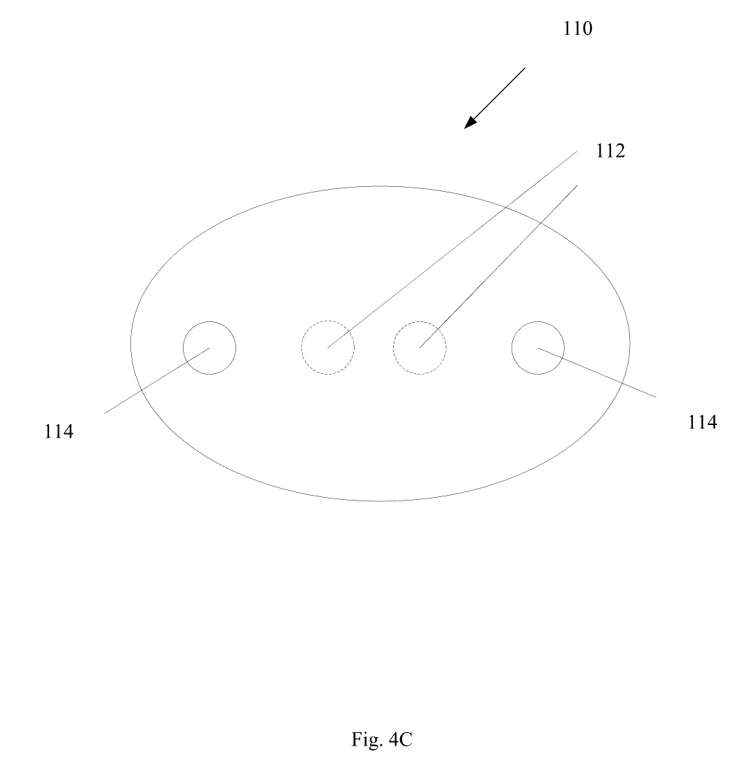

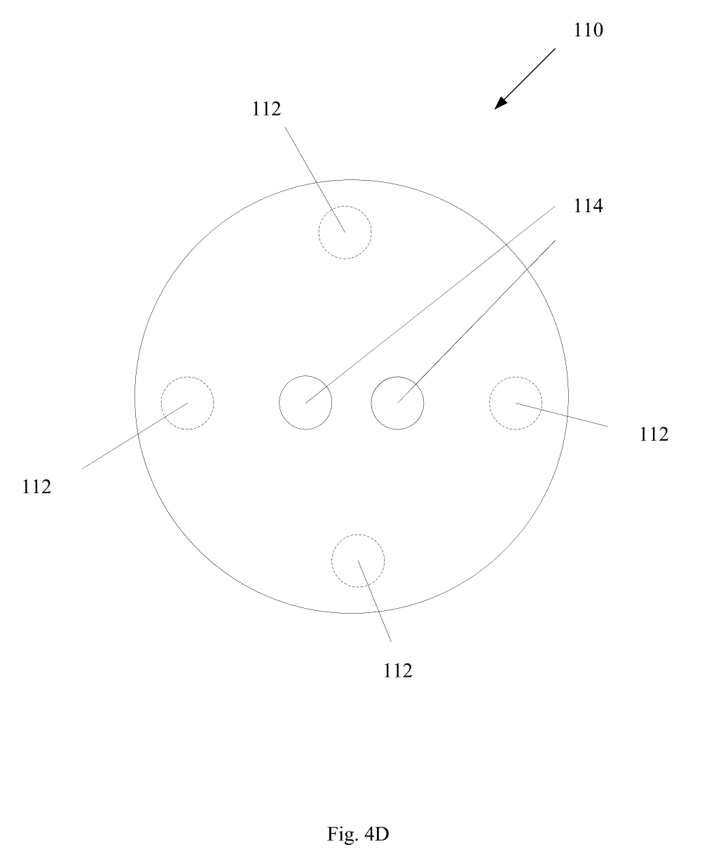

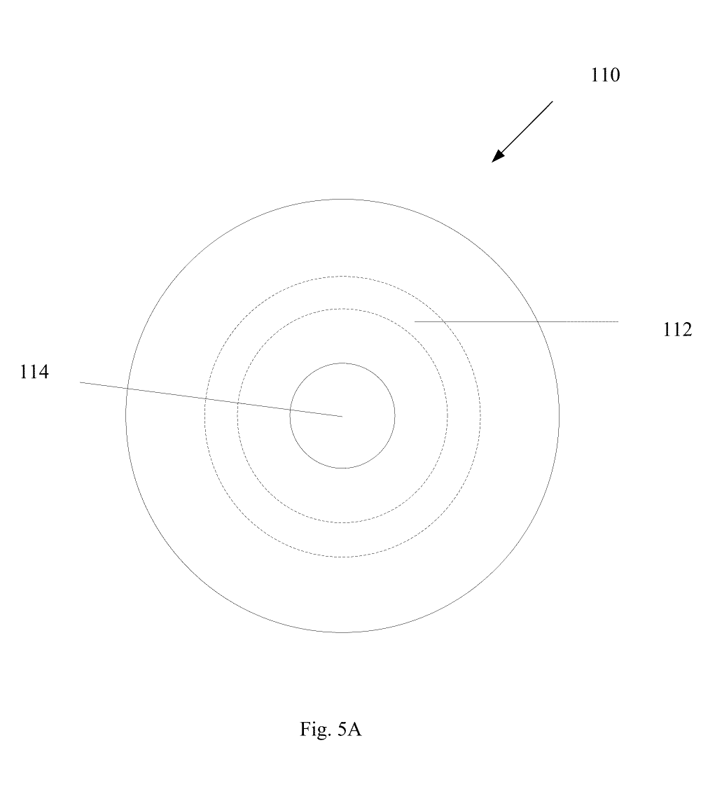

FIGS. 4A-4D illustrate some exemplary configurations for the energy delivery head 110. In some configurations, the energy delivery head 110 can have more than one electromagnetic coil 112. Similarly, the energy delivery head 110 can have any number of other energy sources 114. For TENS, the energy delivery head can have any number of electrodes 114 to deliver TENS so long as there are at least two electrodes 114. The electrodes 114 can be positioned in different configurations on the energy delivery head 110 to provide the desired effect. The energy delivery head 110 can also be any shape or size. For example, as shown in FIGS. 4A and 4B, the electrodes 114 can be positioned in a circle around the electromagnetic coil(s) 112 on a circular energy delivery head 110. Although a circular energy delivery head 110 is illustrated, it will be understood that the energy delivery head 110 can have any other shape. The energy delivery head 110 can be any size or shape suitable for delivering energies to target areas of a user's body. For example, the energy delivery head 110 can be circular, oval, triangular, rectangular, octagonal, or any other shape.

This configuration of four or more electrodes 114 around an electromagnetic coil(s) 112 operates on the same principle as the configuration shown in FIG. 3, but the greater number of electrodes 114 provides an even larger area of therapy for short-term and shallow pain relief while providing deep tissue healing with PEMF from the electromagnetic coil 112. The greater the number of electrodes, the larger the area of therapy provided for short-term and shallow pain relief.

In yet other configurations, as shown in FIGS. 4B and 4C, two or more electromagnetic coils 112 can be positioned between two or more TENS electrodes 114. This configuration operates on the same principle as described above, but provides a larger PEMF therapy area. The greater the number of electromagnetic coils, the larger the area of therapy provided for longer-term, deep tissue healing. In FIG. 4D, four electromagnetic coils 112 are positioned around two electrodes 114.

In conventional TENS devices, electrodes are connected by wires to the main body of the device and the electrodes are also provided with adhesive and are positioned at various locations on the body. These movable electrodes can be cumbersome to use. Embodiments of the wearable energy delivery system 100 described herein has electrodes 114 that are fixed in their locations in the energy delivery head 110 and therefore easier and less cumbersome to use and to move around to different areas of the body to target different problem areas.

As is well known, massage improves blood flow, which can remove metabolic wastes from the body, thereby taking away chemical products from cellular activity in muscles that cause aches. The improved blood flow speeds up the removal of these chemical products from the area and return of the blood that is free of such chemical products. PEMF can also provide better blood flow, slight ionization of the blood, and electric potential in cells to stimulate cellular repair an aid in the healing process.

The energy delivery head 110 of a wearable energy delivery system 100 for delivering TENS (in addition to at least one other type of energy) will have two or more electrodes 114 for contacting the skin of the user.

As noted above, other configurations of the energy delivery head 110 are possible. For example, more than one electromagnetic coil 112 can be provided in the energy delivery head 110, as shown in FIGS. 4B-4D. Combined with different energy sources described above, the energy delivery head 110 can be configured with pairs of PEMF electromagnetic coils 112 to provide either diffused large area therapy or focused small area therapy. If a pair of PEMF electromagnetic coils 112 is positioned next to each other and configured with the same current flow direction (same polarity of magnetic fields), the magnetic therapy area is large and not as concentrated. If a pair of PEMF electromagnetic coils 112 is positioned next to each other and configured with opposite current flow directions (opposite polarity magnetic fields), the magnetic therapy will be provided in a concentrated area between the two opposite polarity PEMF electromagnetic coils.

Thus, if one or more pairs of electromagnetic coils 112 having opposite polarity are provided in the energy delivery head 110 and they are activated at the same time, the electromagnetic coils 112 can provide a focused magnetic field as opposed to a diffused magnetic field, which can be provided by multiple electromagnetic coils 112 having the same polarity and activated simultaneously. It will be understood that, for a pair of electromagnetic coils 112 having opposite polarity and providing a focused magnetic field, the current in the two coils flows in opposite directions (i.e., one clockwise and the other counterclockwise). For electromagnetic coils 112 to provide a large, diffused magnetic field, the electromagnetic coils 112 will have the same polarity and the current in each coil flows in the same direction (i.e., all clockwise or all counterclockwise). Thus, multiple pairs of electromagnetic coils 112 can be configured to have the same polarities or opposite polarities in accordance with the desired therapy.

Multiple PEMF electromagnetic coils 112 can be configured with different energy delivery sequences to provide different therapies. In some embodiments, the energy delivery head 110 can have multiple electromagnetic coils 112 that are activated in sequential or even random order to provide therapy. Since tissue adapts to constant stimulation, it is beneficial to have time varying PEMF energy delivery in different areas. By providing multiple PEMF pulses to the different areas of the tissues at different times, tissue adaptation is reduced or possibly eliminated. Thus, by varying the sequence of activation of the electromagnetic coils 112, saturation of particular tissue areas may be minimized or even eliminated.

As shown in FIGS. 4B and 4C, the one or more electromagnetic coils 112 can be positioned in the center of the working surface relative to two or more electrodes 114, which are positioned around the periphery. The electromagnetic coil(s) 112 in the center can deliver PEMF to provide healing therapy targeted at the problem area while the electrodes 114 can be used to deliver energy for blocking pain on the periphery. As discussed above, TENS can be used for blocking pain sensations. Similarly, other energies, such as far infrared, can also be used to block pain. PEMF, in addition to providing healing therapy, can also block pain depending on the strength of the electromagnetic coil(s).

In other embodiments, such as the one illustrated in FIG. 4D, two or more electrodes 114 can be positioned in the center of the working surface relative to one or more electromagnetic coils 112. This configuration of positioning TENS electrodes 114 between PEMF electromagnetic coils 112 and coils provides deeper healing therapy on the larger area scale with focused shallow therapeutic effect in the center of the larger PEMF area. This configuration provides short-term relief from the TENS to the shallow surface area and longer-term healing of area due to PEMF. This configuration of positioning the TENS electrodes between PEMF can be configured in multiple ways.

In other embodiments, PEMF is combined with another type of energy. In some embodiments, PEMF is combined with heat energy, such as near or far infrared. Such a combination provides a soothing heat effect to the skin while also providing deep tissue healing energy from PEMF. The heat source can be either one or can be more than one and can be shaped in any way to provide a soothing heat effect to the skin. For example, if a large area of soothing heat effect is desired, multiple heat sources can be provided in an arrangement to cover the area or a single heat source that has the shape of the targeted area can be provided. If a smaller area of heat soothing is desired, one or more small heat sources shaped in accordance with the problem area can be provided. The heat source(s) can be positioned anywhere on the energy delivery head 110. For example, the heat source(s) can replace the electrodes in the examples described above. Thus, in FIGS. 3 and 4, features designated by the reference numeral 114 can represent a different type of energy source, such as a heat source.

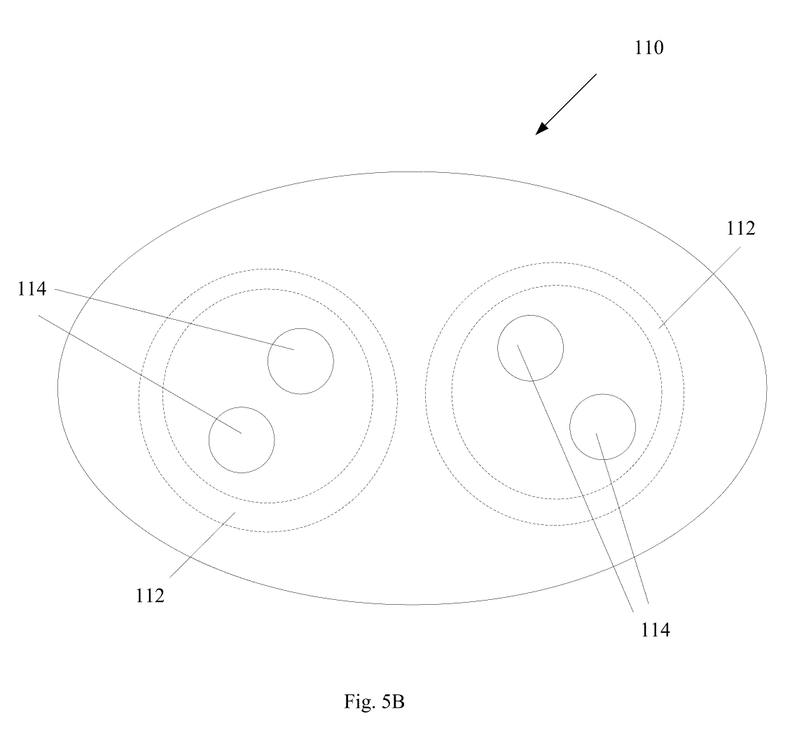

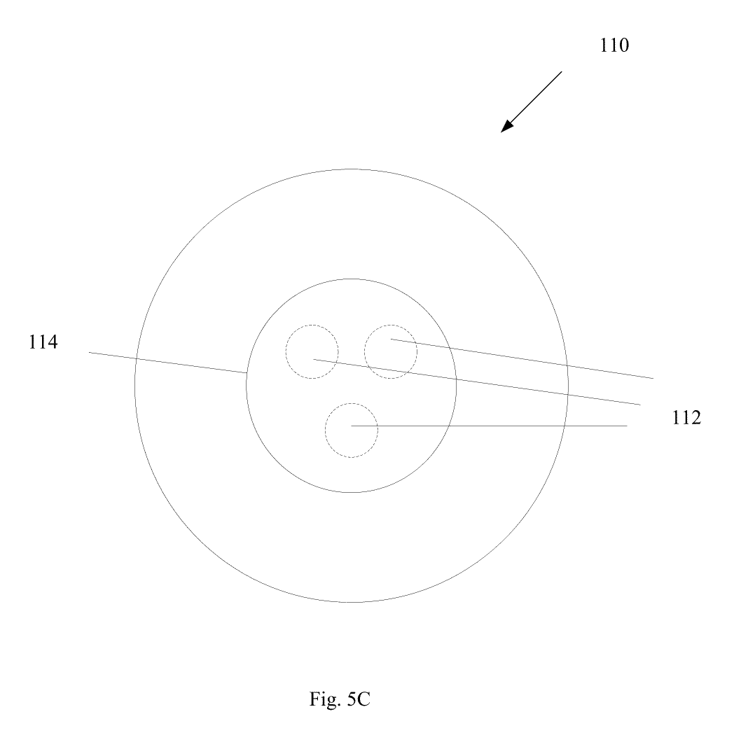

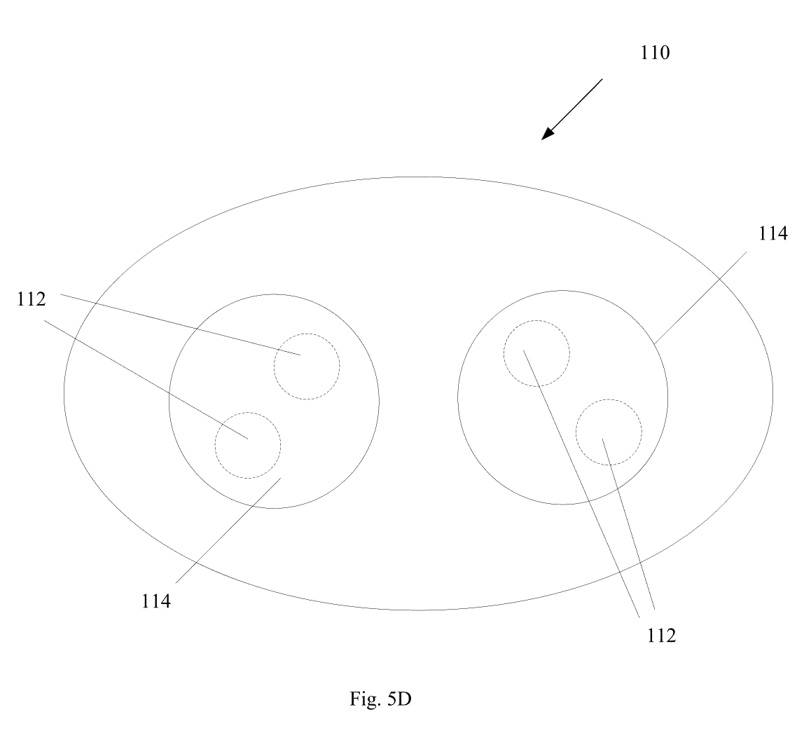

FIGS. 5A-5D illustrate different configurations of an energy delivery head having a PEMF coil 112 and another energy source 114 (e.g., ESP, TENS, heat, etc.) nested within one another. For example, in the embodiment shown in FIG. 5A, a different energy source 114 is nested within the electromagnetic coil 112. In the embodiment shown in FIG. 5B, one or more other energy sources 114 are located within the one or more electromagnetic coils 112. In the embodiment shown in FIG. 5C, one or more electromagnetic coils 112 are located within another energy source 114. In the embodiment shown in FIG. 5D, one or more electromagnetic coils 112 are located within one or more other energy sources 114.

Thus, it will be understood that an energy delivery head 110 for delivering a combination of PEMF and heat energy can be configured in multiple ways, depending on the effect desired. According to one embodiment, an electromagnetic coil 112 can be provided in the center between heat source(s) 114. This configuration will provide a soothing effect to the skin with increased blood flow to the area due to the higher temperature (from the heat source 116) affecting shallow areas of the skin or muscle and PEMF will provide additional blood flow and deeper healing due to the penetrating property of PEMF.

The wearable energy delivery system 100 can be configured in a variety of different ways. According to one embodiment, the wearable energy delivery system 100 is an integrated system, with the energy delivery head 110, the power control unit 120, and the user interface 130 all integrated as a single unit. The wearable energy delivery system 100 can be battery operated (rechargeable or otherwise) and/or powered wirelessly by another device, such as a computer or a smartphone.

Similarly, the wearable energy delivery system 100 can be wirelessly controlled by a device, such as a smartphone, tablet, computer, smart watch, etc. In this embodiment, the energy delivery head(s) 110 are integrated with the wearable item and user interface 130 is part of the controlling device (e.g., smartphone). The energy delivery head(s) 110 can be operably connected to the power control unit 120 either wirelessly or by wires 122. If the energy delivery head(s) are connected by wires 122 to the power control unit 120, the wires 122 can be woven into the fabric of the wearable items, hidden in or along seams, covered by piping, and/or integrated with the fabric in a decorative pattern. It will be understood that the wires 122 are insulated. The power control unit 120 can be placed in a pocket of the wearable item or in a pouch designed to hold the power control unit 120. In an alternative embodiment, the energy delivery head 110 and the power control unit 120 can be integrated in a single enclosure that is integrated with or otherwise attached to the wearable item. In some embodiments, the power control unit 120 can be placed in a pocket or pouch of the wearable item along with a power supply unit, such as a battery pack.

According to another embodiment of the system 100, each of the three components is a separate subsystem. In this embodiment, the subsystems can be operably connected with one another either in a wireless manner (e.g., Wi-Fi, Bluetooth, Near Field Communication (NFC), etc.) or by wires, or a combination thereof.

According to other embodiments, the three components are separated into two subsystems. For example, the energy delivery head 110 and the power control unit 120 are integrated as a subsystem and the user interface 130 is on a separate subsystem. According to another example, the power control unit 120 and the user interface 130 are integrated as a subsystem and the energy delivery head 110 itself is a separate subsystem. In yet another example, the energy delivery head 110 and the user interface 130 are integrated together as a subsystem and the power control unit 120 is a separate subsystem. For the configurations described above, the two subsystems can be operably connected either wirelessly (e.g., Wi-Fi, Bluetooth, NFC, etc.) or by wires, or a combination thereof.

In some embodiments, the energy delivery head 110 is flexible to fit body contours. Flexible elastomers, such as, silicone, Sentoprene™, Nitrile, Neoprene, ethylene propylene diene monomer (EPDM) rubber, and other synthetic elastomers, can be used to form the outer surfaces of the energy delivery head 110. Alternatively, the energy delivery head 110 can be rigid. Some materials that are suitable for a rigid energy delivery head include ABS, polycarbonate, and other thermal plastics. It will be noted that a rigid material for the energy delivery head 110 can be beneficial, for example as insoles for foot therapy.

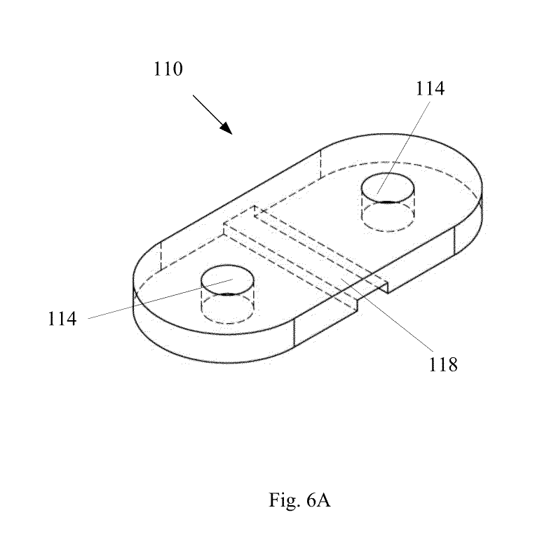

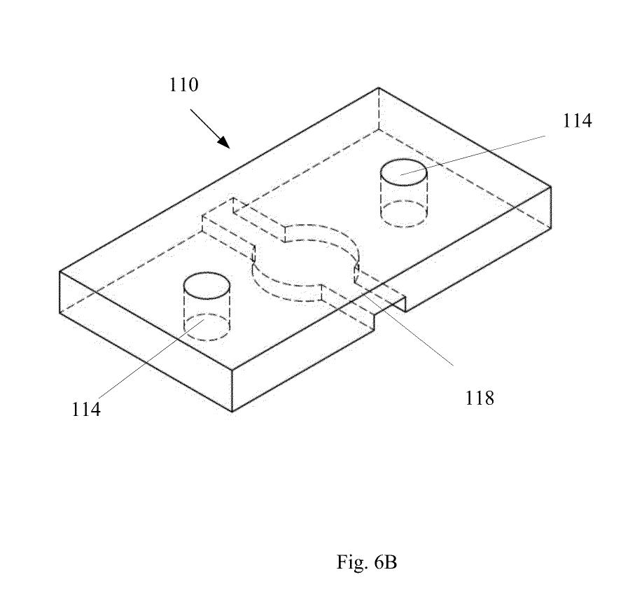

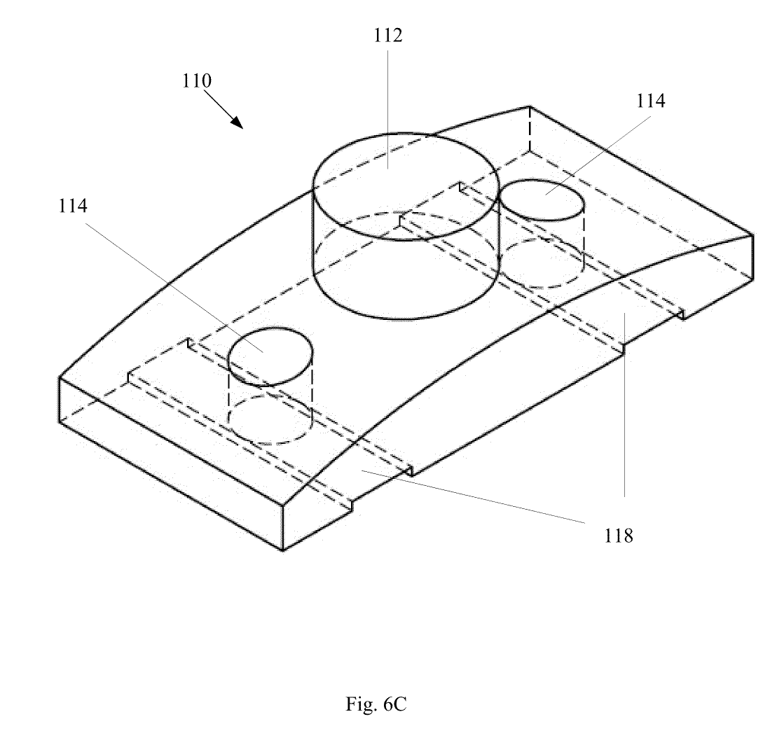

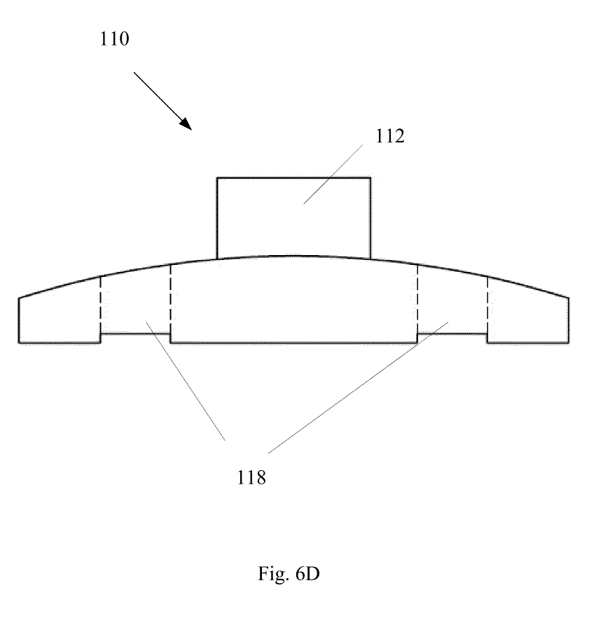

As shown in FIGS. 3A, 3B, and 6A-6B, the energy delivery head 110 can also be provided with flex points or strips 118 to allow the energy delivery head 110 to bend to better fit the contours of the portion of the body to which it is applied. It will be understood that the flex points or strips 118 can be positioned anywhere on the energy delivery head 110 and in any orientation. For example, in the embodiment shown in FIGS. 3A and 3B, the flex points or strips 118 are positioned on either side of the electromagnetic coil 112 and on both the top surface and the bottom or working surface of the energy delivery head 110. In the embodiments shown in FIGS. 6A and 6B, the flex strips 118 extend out from the electromagnetic coil (not shown for simplicity in FIGS. 6A and 6B) in the center of the energy delivery head 110. In other embodiments, the flex points or strips can, for example, be oriented lengthwise along the energy delivery head 110 or radiating out from the center of the energy delivery head 110. The flex points 118 are formed of suitable flexible materials, such as low durometer elastomers. It should also be noted that the working surface can be either substantially flat, as shown in FIGS. 6A and 6B, or it can be curved, as shown in FIGS. 6C and 6D, to fit particular body contours. Alternatively, in some embodiments, the electromagnetic coil 112 can also protrude from the working surface, as shown in FIGS. 6C and 6D.

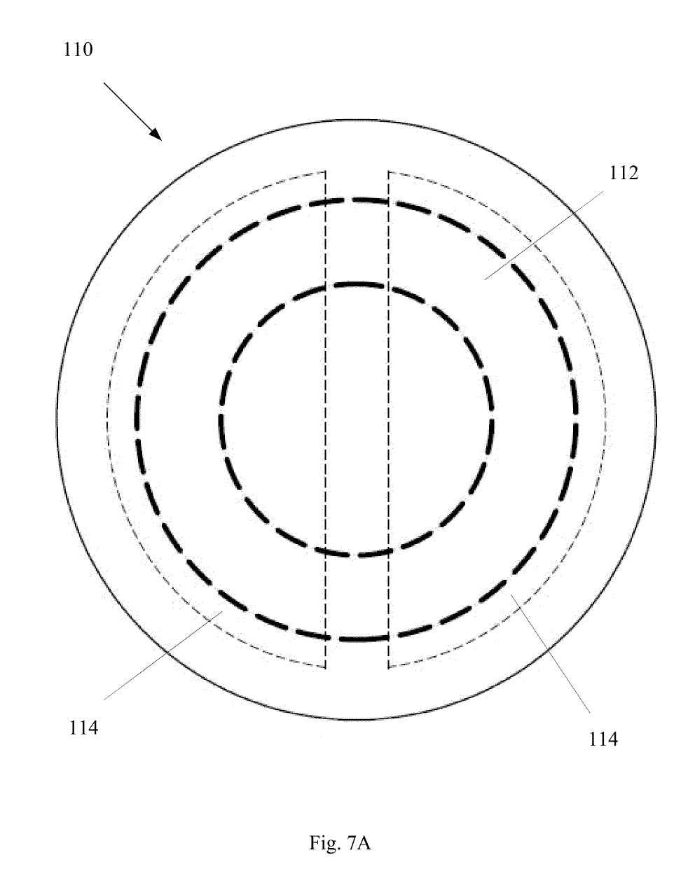

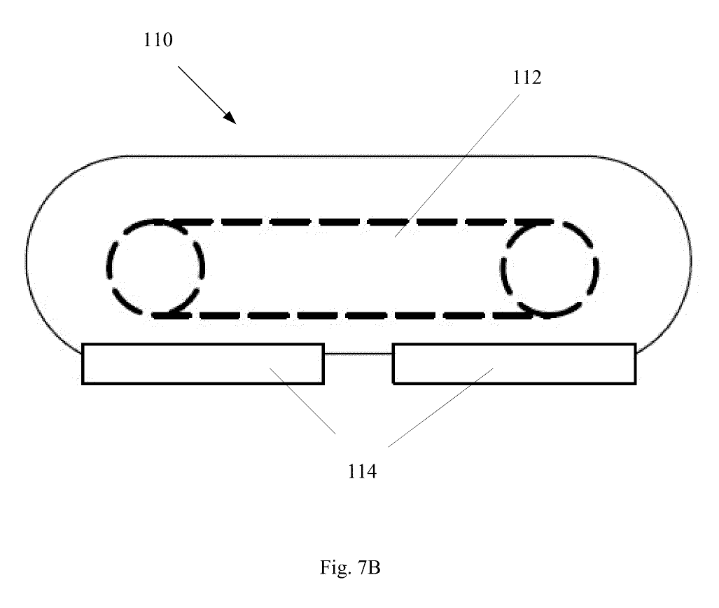

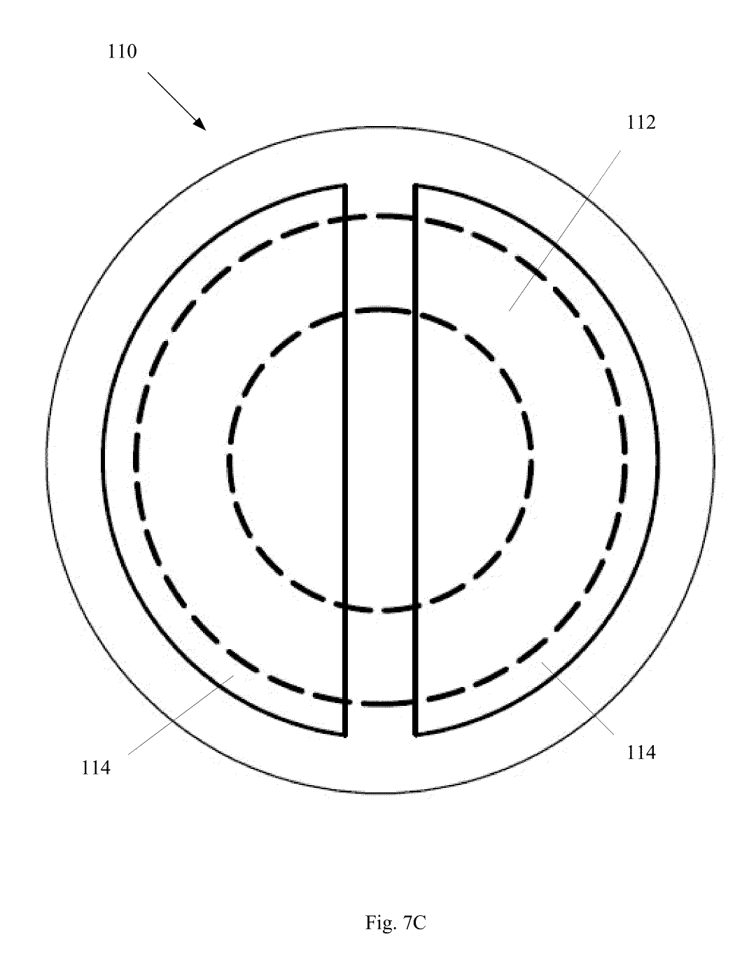

FIGS. 7A-7C show an energy delivery head 110 having an electromagnetic coil 112 stacked over electrodes 114 in accordance with an embodiment. FIG. 7A is a top view of the energy delivery head 110 showing the embedded electromagnetic coil 112 in bold dashed lines, and the two electrodes 114 in dashed lines. As shown in the side view of FIG. 7B, the electromagnetic coil 112 is positioned over the two electrodes 114. FIG. 7C is a bottom view of the energy delivery head 110 showing the two electrodes 114 (shown by solid lines) at the working surface.

The electromagnetic coil 112 for delivering the PEMF can either have a magnetic core or it can be hollow without a core (air). The energy delivery head 110 can also have more than one electromagnetic coil 112, as noted above. The one or more electromagnetic coils 112 can provide either a standard magnetic field or paired opposite polarity electromagnetic coils can provide a focused magnetic field.

The power control unit 120 provides power to the energy delivery head 110 and could provide power to the user interface 130. The power control unit 120 can be operably connected to the energy delivery head 110 and the user interface 130 in different ways. According to one embodiment, the power control unit 120 is hard-wired to the energy delivery head 110 and/or the user interface 130. According to another embodiment, the power control unit 120 is a unit that can be separated from the energy delivery head 110 and/or the user interface 130, and each of the components is operably connected to another by plugging one component into another or by wires. The components can also be operably connected to one another in a wireless manner

Like the energy delivery head 110, the power control unit 120 can also be flexible or rigid. The energy source for the power control unit 120 can be either a battery (for a portable unit), other portable energy source (such as solar), or it can have a plug-in electrical connection. In some embodiments, the power control unit 120 has an enclosure that can include a variety of circuits for power delivery, power control, safety control, communication with the user interface 130, communication with the energy delivery head 110, and integrated circuit chips.

According to an embodiment, the user interface 130 has a display, buttons, keyboard, control knobs, and/or other types of user control interface to allow a user to control the wearable energy delivery system 100. The user interface 130 allows a user to select the type(s) of energy the wearable energy delivery system 100 delivers. In some embodiments, the user can also use the user interface 130 to determine the intensity of each type of energy to be delivered by the wearable energy delivery system 100. The power control unit 120 and the user interface 130 can also have wireless capability and be controlled remotely by other devices, such as smart devices (e.g., smartphone, tablet, etc.), computer, and remote control.

The user interface 130 is provided to control the power control unit 120 and energy delivery head 110. Like the energy delivery head 110 and power control unit 120, the user interface 130 can be either flexible or rigid. As noted above, the user interface 130 can also have a display, buttons, keyboard and/or control knobs or it can be integrated with another device, such as a smartphone, computer, tablet, smart watch, etc.

Any of the components 110, 120, 130 of the wearable energy delivery system 100 can be configured to be permanently (e.g., sewn-in or woven into fabric) or removably (e.g., attached using snaps, buttons, Velcro®, buckles, placement in pouches, etc.) attached to a wearable item, such as clothing, straps, and shoes. For example, it may be desirable to provide athletic wear with an energy delivery system 100 to provide a reenergizing effect to the user, which can be desirable during strenuous exercise. It will be noted that PEMF does not need skin contact, but TENS does require skin contact. Thus, tight athletic wear (e.g., bra, running tights, yoga pants, straps, etc.) that makes sufficient contact with skin is suitable for integration with a TENS delivery system 100. As PEMF does not require skin contact to work, a PEMF delivery system 100 can be integrated in loose-fitting clothing. Other energy sources, such as ESP, requires skin contact whereas other energy sources, such as infrared does not require skin contact.

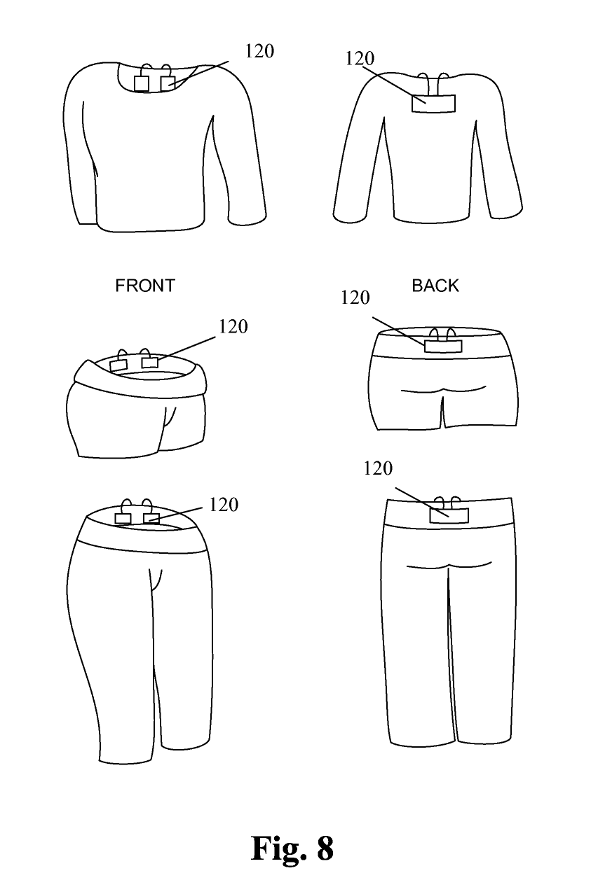

Depending on the configuration, either the entire system 100 can be permanently or removably attached to the wearable item or selected subsystems can be permanently or removably attached to the wearable item, or any combination thereof. For example, in one embodiment, one or more energy delivery heads 110 are permanently or removably attached to the wearable item and a combined power control unit 120 and user interface 130 unit is operably connected to the energy delivery head 110 either wirelessly or by wires. If the combined power control unit 120 and user interface 130 unit is wirelessly connected to the energy delivery head(s) 110, it does not need to be attached to the wearable item and can be a remote unit. If the combined power and control unit 120 and user interface 130 unit is connected to the energy delivery head(s) 110 by wires 122, it can either be permanently or removably attached to the wearable item or it can be simply placed in a pocket or a pouch of the wearable item. FIG. 8 shows different embodiments of the energy delivery head(s) 110 and power control units 120 on a wearable item. Although illustrated as a power control unit 120 in FIG. 8, it will be understood that the power control unit 120 can be combined in a single enclosure with a user interface 130 in some embodiments. The power control unit 120 and/or user interface 130 can also be located away from the energy delivery head(s) 110 and/or the wearable item to provide convenience and/or comfort for the user.

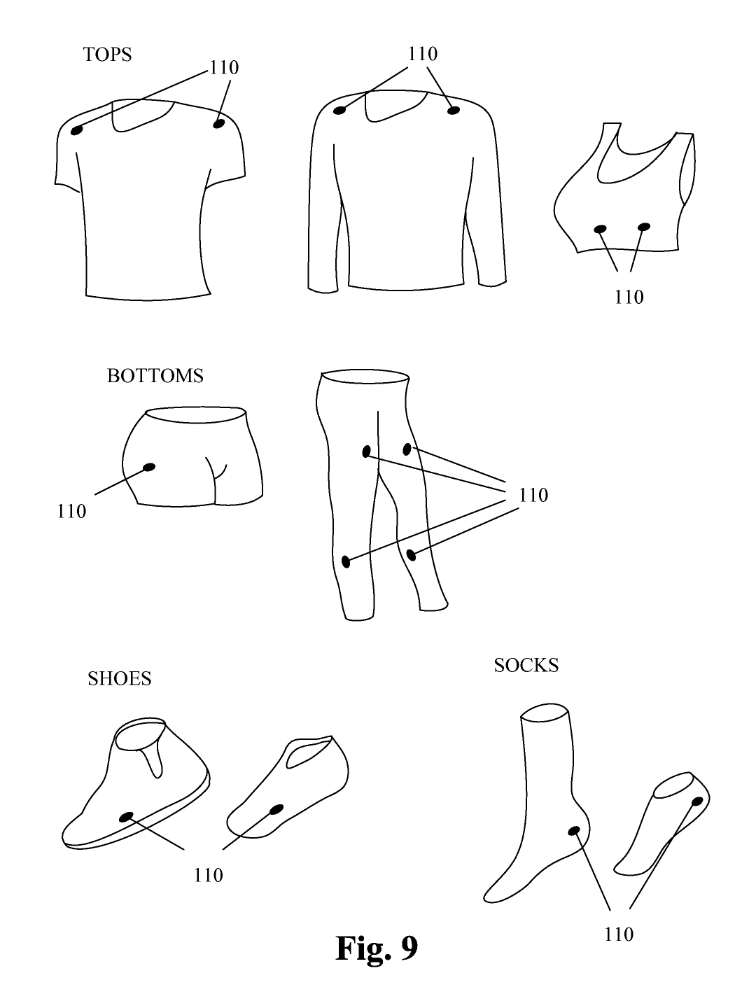

As shown in FIG. 9, the energy delivery heads 110 can be integrated anywhere on a variety of wearable items. As noted above, the energy delivery head(s) 110 can be permanently integrated in the wearable item or can be removably attached, using Velcro®, snaps, buttons, straps, pockets, pouches, and the like. If the energy delivery heads 110 are removably attached to the wearable item, the energy delivery heads 110 can be moved, as desired, to different part of the wearable item to provide therapy to different parts of the user's body to target different problem areas.

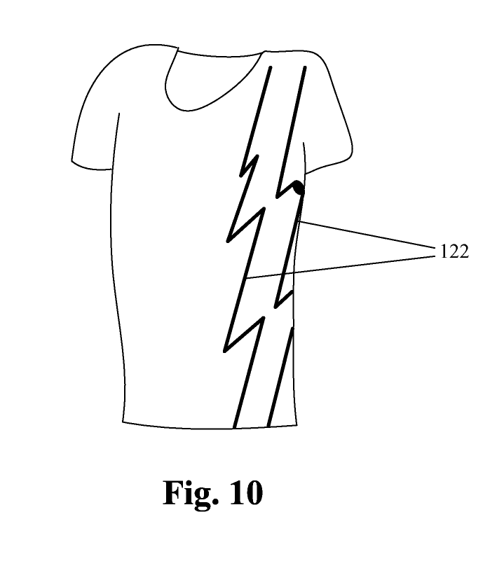

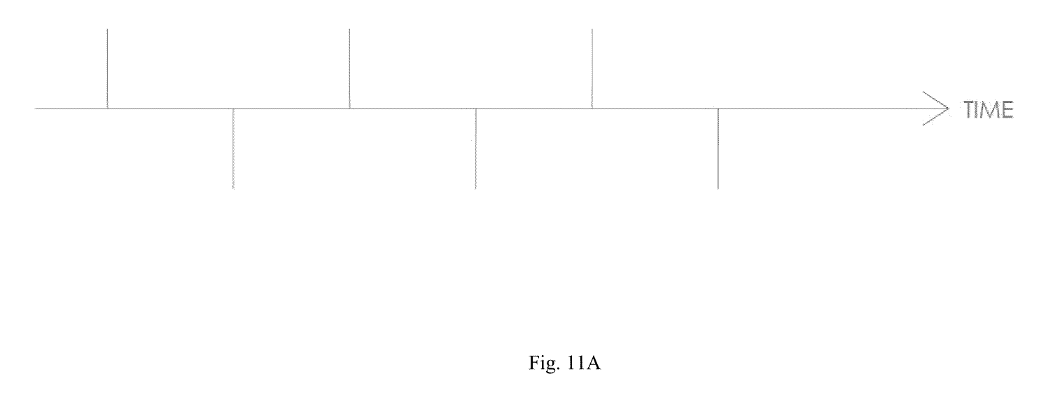

If wires are used to operably connect components, the wires can be hidden in the wearable item. For example, the energy delivery head 110 and/or the wires can be woven into fabric, sewn into or otherwise attached along seams of a garment, or hidden under straps or piping. Alternatively, wires 118 can create a decorative element of the wearable item, as shown in FIG. 10, which shows an example of decorative wiring in clothing in which the wires 118 form a decorative lightning bolt on a shirt.

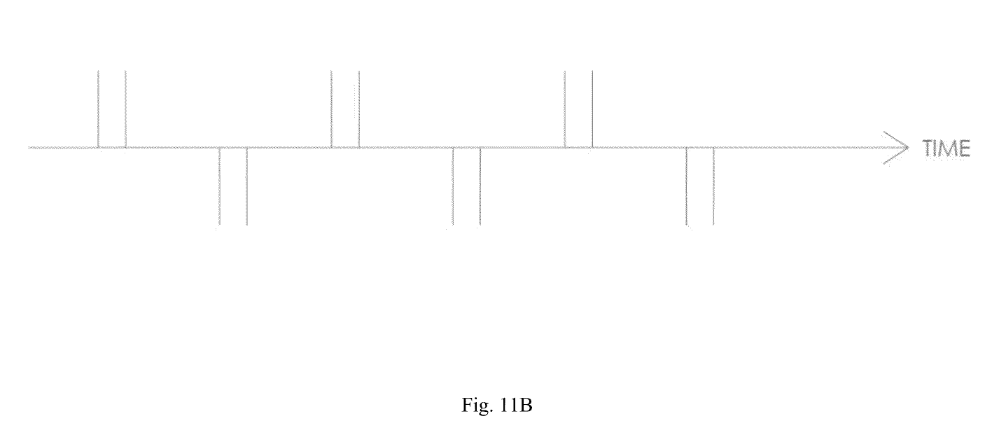

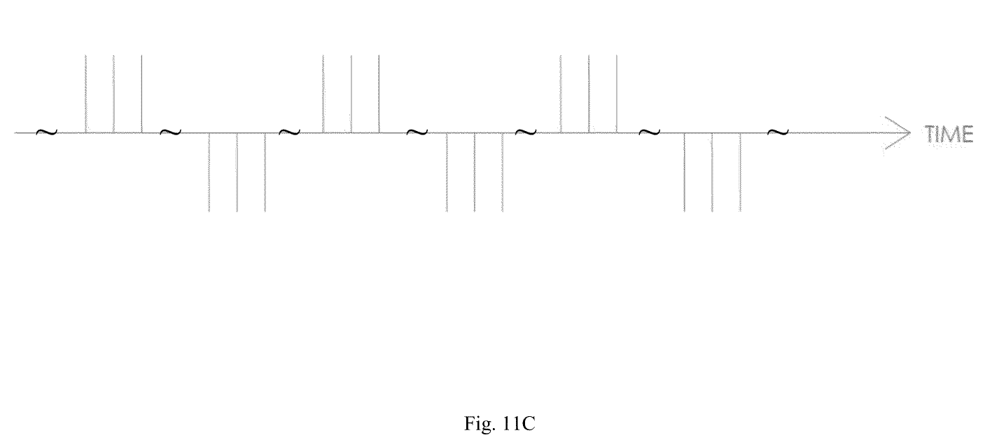

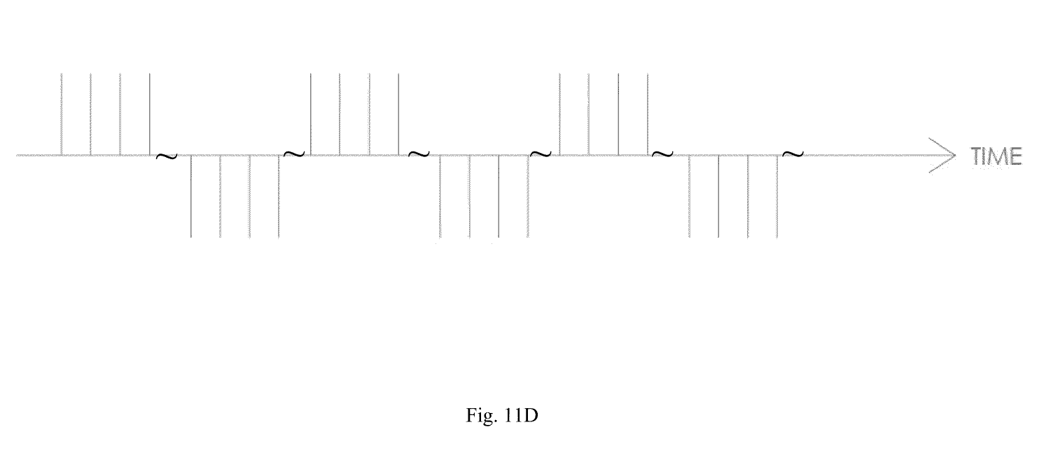

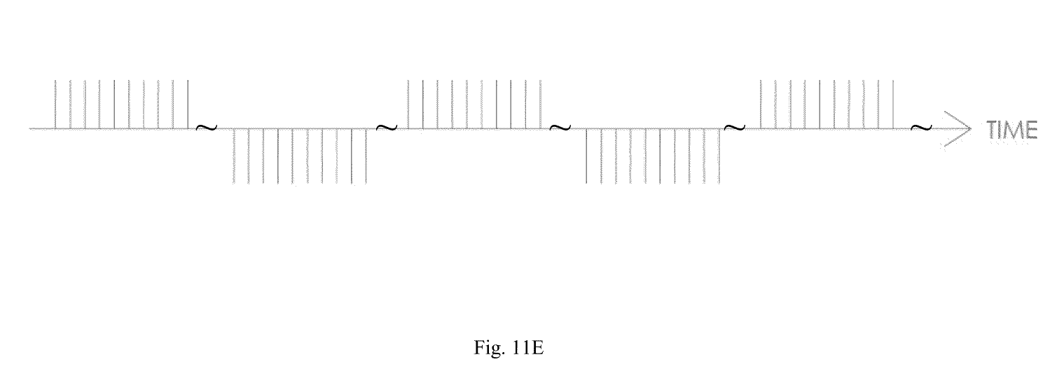

For effective therapy of aches and pain, delivery of PEMF energy should be varied over time because of the human body's ability to ignore constant stimulus. To prevent tissue from adapting to the energy that is being delivered, effective energy delivery patterns can be developed and these patterns can be varied over the course of time. With reference to FIGS. 11A-11E, different embodiments of energy delivery patterns, with both positive and negative energy, are described. The energy delivery varies the frequencies over different time periods, mixing higher frequencies with lower frequencies over short and long periods of time. The wearable energy delivery system 100 can be configured to deliver a program that includes different energy delivery patterns, such as those shown in FIGS. 11A-11E or other energy delivery patterns to vary the energy delivered so that tissue does not adapt to any particular pattern.

In some embodiments, the pulse width of an energy delivery pulse is about 50 to 150 microseconds with a more preferable range of about 85 to 115 microseconds (μs) and more preferably about 100 μs. Each pulse is shown as a line in the energy delivery pattern in FIGS. 11A-11E, and has a pulse width of about 50 to 150 μs (or more preferable about 85 to 115 μs). The energy delivery head can be configured to generate the different electrical waveforms or patterns shown in FIGS. 11A-11E.

According to the energy delivery pattern shown in FIG. 11A, single pulses are delivered with alternating polarity, and the frequency is in a range of about 0.1 to 17 Hz, and more preferably in a range of about 0.1 to 8 Hz. It will be understood that the energy delivery profile shown in FIG. 11A is only an example and that the polarity does not need to alternate after each pulse. For example, the polarity can be alternated after two or more pulses (e.g., deliver two positive pulses and then deliver two negative pulses, or deliver two positive pulses and then deliver three negative pulses followed by three positive pulses and then followed by two negative pulses) or the pulses can be only positive pulses or only negative pulses.

The energy delivery pattern shown in FIG. 11B includes two or more frequencies within the pattern. A burst of two quick pulses with a frequency of about 50 to 150 Hz (or a more preferable frequency of 80 to 120 Hz) is delivered, as shown in FIG. 11B. Although not shown in FIG. 11B, pulses between each burst of two or more quick pulses have lower frequencies of about 0.1 to 17 Hz, or more preferable about 0.1 to 8 Hz, or even more preferably about of 0.1 to 4 Hz. In a particular embodiment, the frequency of pulses between the bursts is about 0.5 to 2 Hz. In this example, the polarity of the pulses is not alternated between the bursts.

The energy delivery patterns of FIGS. 11C-11E are similar to the energy delivery pattern of FIG. 11B. Each of the energy delivery patterns includes a burst of multiple quick pulses. As with the pattern of FIG. 11B, the bursts alternate polarity. FIG. 11C shows bursts of three quick pulses each. FIG. 11D shows bursts of four quick pulses each, and FIG. 11E shows bursts of five quick pulses each.

An energy delivery program of the wearable energy delivery system 100 could have a duration of 10 to 45 minutes, or more preferably 25 to 35 minutes. Each program can have a combination of energy delivery patterns, such as those shown in FIGS. 11A-11E, and their variations.

In one example, an energy delivery program of 30 minutes can include multiple segments of different energy patterns. It will be understood that different variations of different energy delivery patterns can be used in different sequences for an energy delivery program. Each energy pattern can repeat itself for a given period of time. Below is an exemplary 30 minute program that could be delivered by the device:

| Minutes | Pattern |
|---|---|
| 1-3 | Pattern of FIG. 11A, but with positive pulses only |
| 3-6 | Pattern of FIG. 11A with positive and negative alternating |
| | pulses as shown |
| 6-9 | Pattern of FIG. 11A, but with negative pulses only |
| 9-12 | Pattern of FIG. 11C as shown |
| 12-15 | Pattern of FIG. 11B, but with negative pulses only |
| 15-18 | Pattern of FIG. 11B, but with positive pulses only |
| 18-21 | Pattern of FIG. 11D, as shown |
| 21-24 | Pattern of FIG. 11A with three positive pulses followed by two |
| | negative pulses followed by two positive pulses followed by |
| | three negative pulses |
| 24-27 | Pattern of FIG. 11A with five negative pulses followed by four |
| | positive pulses followed by three negative pulses |
| 27-30 | Pattern of FIG. 11A, as shown |

Although only a few embodiments of the invention have been described in detail, it should be appreciated that the invention may be implemented in many other forms without departing from the spirit or scope of the invention. It should be apparent that the described energy delivery devices can be used in a wide variety of applications. In view of all of the foregoing, it should be apparent that the present embodiments are illustrative and not restrictive and the invention is not limited to the details given herein, but may be modified within the scope and equivalents of the appended claims.

1. A wearable pulsed electromagnetic energy delivery device, comprising:
an article of clothing;
an energy delivery head configured to generate a pulsed electromagnetic field (PEMF), wherein the energy delivery head is integrated with the article of clothing, and wherein the energy delivery head comprises multiple layers of flexible material, wherein each of the flexible material layers includes at least one layer of trace or coil wire, wherein the at least one layer of trace or coil wire in a layer of flexible material is separate from a layer of trace or coil wire in another layer of flexible material;
a power supply unit arranged to be carried by the article of clothing; and
at least one wire that couples the power supply unit to the energy delivery head, wherein the at least one wire is integrated with the article of clothing; and
wherein the wearable pulsed electromagnetic energy delivery device is arranged to deliver therapeutic pulsed electromagnetic energy to a wearer of the article of clothing without requiring power from a device not carried by the article of clothing.

2. The device of claim 1, wherein the energy delivery head is permanently attached to the article of clothing.

3. The device of claim 2, wherein the energy delivery head is sewn into fabric of the article of clothing.

4. The device of claim 1, wherein the energy delivery head emits PEMF in combination with another type of energy.

5. The device of claim 1, wherein the at least one wire that couples the power supply unit to the energy delivery head is hidden in seams of the article of clothing.

6. The device of claim 1, wherein the at least one wire that couples the power supply unit to the energy delivery head is attached to fabric of the article of clothing in a decorative pattern.

7. The device of claim 1, wherein the at least one wire that couples the power supply unit to the energy delivery head is woven into fabric of the article of clothing.

8. The device of claim 1, wherein the flexible material layers are bonded together to form one single piece of flexible material.

9. The device of claim 1, wherein the wearable pulsed electromagnetic energy delivery device is controlled wirelessly by an external device.

10. A system, comprising:
a wearable pulsed electromagnetic energy delivery device including an energy delivery head configured to generate a pulsed electromagnetic field (PEMF) and a power control unit arranged to generate an electrical waveform used by the energy delivery head to generate the pulsed electromagnetic field wherein the wearable pulsed electromagnetic energy delivery device is arranged to deliver therapeutic pulsed electromagnetic energy to a wearer of the pulsed electromagnetic energy delivery device without requiring power from an external device not carried by the pulsed electromagnetic energy delivery device, wherein the energy delivery head comprises multiple layers of flexible material, wherein each of the layers of flexible material includes an electromagnetic coil that has multiple separate layers of trace or coil wire embedded therein; and
a mobile user interface executable on a computing device separate from the wearable device, wherein the mobile user interface is arranged to utilize resources of the computing device to communicate wirelessly with the power control unit and is configured to permit a user to direct operation of the wearable pulsed electromagnetic energy delivery device via the computing device.

11. The system of claim 10, wherein the wearable pulsed electromagnetic energy device is operably connected to the power control unit by at least one wire woven into fabric of a wearable item.

12. The system of claim 11, wherein the at least one wire is removably attached to the wearable item.

13. The system of claim 11, wherein the at least one wire is attached along seams of the wearable item.

14. The system of claim 11, wherein the energy delivery head is removably attached to the wearable item.

15. The system of claim 14, wherein each layer of flexible material includes an electromagnetic coil.

16. The system of claim 11, wherein the energy delivery head delivers a different type of energy in addition to the pulsed electromagnetic energy.

17. The system of claim 11, wherein the wearable pulsed electromagnetic energy delivery device is a single integrated unit.

18. The system of claim 11, wherein the energy delivery head is configured to direct the pulsed electromagnetic field toward a user body part adjacent the wearable device when the wearable device is worn by a user and the energy delivery head comprises multiple flexible material layers that are loosely fastened together.

19. A wearable energy delivery device, comprising:
an energy delivery head configured to emit at least two different types of energy selected from the group consisting of pulsed electromagnetic field (PEMF) energy, pulsed electrical energy, and heat energy, wherein the energy delivery head comprises multiple layers of flexible material, wherein at least one layer of flexible material includes multiple separate layers of trace or coil wire; and
a power control unit operably connected to the energy delivery head by at least one wire, wherein the energy delivery head and the wire are attached to footwear.

20. The wearable energy delivery device as recited in claim 19, wherein the wearable energy delivery device is arranged to deliver therapeutic pulsed electromagnetic energy to a wearer of the energy delivery device without requiring power from an external device not carried by the energy delivery device.

21. The wearable energy delivery device of claim 19, wherein the power control unit includes a power controller arranged to generate an electrical waveform used by the energy delivery head to generate a pulsed electromagnetic field.

22. The wearable energy delivery device of claim 19, wherein the power control unit includes a battery that supplies power to the energy delivery head.

23. A wearable energy delivery system, comprising:
an energy delivery head configured to emit at least two different types of energy, wherein the energy delivery head is integrated with a wearable item, and wherein the energy delivery head comprises multiple layers of flexible material, wherein at least one layer of flexible material includes an embedded electromagnetic coil comprising multiple lavers of trace or coil wire that are separate from one another; and
a power control unit operably connected to the energy delivery head by a wire, wherein the wire is attached to the wearable item.

24. The wearable energy deliver system of claim 23, wherein the wearable item is a shoe.
